(19) United States
(12) Patent Application Publication
(10) Pub. No.: US 2003/0212052 A1
(43) Pub. Date: Nov. 13, 2003

(54) Use of ursdeoxycholic acid for potentiation of the phototoxic effect of photodynamic therapy

(76) Inventors: David Kessel, Birmingham, MI (US); John Reiners Jr, Franklin, MA (US)

Correspondence Address: Kenneth I Kohn, Kohn & Associates, Suite 410, 30500 Northwestern Highway, Farmington Hills, MI, 48334, US

(21) Appl. No.: 10/257,742

(22) PCT Filed: Apr. 20, 2001
(86) PCT No.: PCT/US01/12813

(51) Int. Cl. A61K 31/57; A61K 31/409; A61K 31/555

(52) U.S. Cl. 514/171; 514/185; 514/410

(57) ABSTRACT

A method of potentiating the phototoxicity of photodynamic therapy by co-administering a photosensitizing agent with a photodynamic potentiator from the group consisting of uridioxicolic acid analogs and conjugates thereof having the photo-toxicity potentiating effect and allowing for retention of the co-administered agent and acid in the target tissue. The target tissue is then irradiated. A tool for potentiating apoptosis consists of a photosensitizing agent and a non-toxic photodynamic potentiator. Further, a method of potentiating drug induced apoptosis is provided by administering a photosensitizing agent and then decreasing the threshold of responsiveness of a target tissue to a photokilling effect of the photosensitizing agent.

| DRUGS(S) | COLONIES | % CONTROL |
|---|---|---|
| CONTROL | 75 ± 5 | 100 |
| UDCA 10-10 μM | 74 ± 14 | 100 ± 18 |
| SnET2 2 μM | 39 ± 16 | 52 ± 21 |
| SnET2 2 + 10 μM UDCA | 12 ± 5 | 20 ± 7 |
| 20 μM | 9 ± 5 | 12 ± 6.5 |
| 50 μM | 2.6 ± 1.5 | 3.5 ± 2 |
| 100 μM | 1 ± 0.7 | 1.3 ± 1 |

### GRANT INFORMATION

Research in this application was supported in part by a grant from the National Institutes of Health (Contract No. CA-23378). The Government has certain rights in the invention.

### BACKGROUND OF THE INVENTION

1. Field of the Invention

The present invention relates to photodynamic therapy and tools used for photodynamic therapy and the initiation of apoptosis. More specifically, the present invention relates to a method of potentiating phototoxicity of photodynamic therapy for use in the treatment of such diseases as cancer, age-related macular degenerization of the RI, and for the treatment of atherosclerotic plaque in arteries.

2. BACKGROUND ART

Photodynamic therapy (also referred to as PDT, photoradiation therapy, phototherapy or photochemotherapy) is a procedure involving photosensitization of tissues by any of a series of agents with a porphyrin-like structure. The term “photosensitization” refers to a sensitivity exhibited by tissues having an effective concentration of a photoactive agent retained therein through the exposure of light which causes intracellular disruption and potentially a cell killing effect, often referred to as photokilling.

Porphyrins are derivatives of the parent tetrapyrrole compound, porphyrin. They are named and classified on the basis of their side chain constituents. The porphyrin ring is present in many heme enzymes and proteins and is also found in chlorophylls of green plant cells.

For reasons yet to be completely explored, the effective agents utilized in photodynamic therapy localize in neoplastic tissues. Various other agents also show an affinity for atherosclerotic plaques and/or a class of retinal blood vessels that lead to macular degeneration.

Upon irradiation with visible light at a suitable wave length, the photoactive agents catalyze the conversion of oxygen dissolved in the surrounding millue through a highly reactive product, singlet molecular oxygen. These agents can bring about the destruction of cellular organelles in the immediate vicinity. Singlet oxygen is quenched by the first biological molecule that it encounters.

A variety of agents can also quench singlet oxygen. For example, vitamin E and glutathione are effective quenchers of singlet oxygen. High levels of these agents can therefore impair a photodynamic response. Other drugs can promote the photokilling. These drugs include the protein kinase C inhibitors, staurosporin and chelerythrine. These agents are toxic and cannot be used in the clinic.

Recently, a major review was published in Photodynamic therapy by most of the leaders in the field (Photodynamic Therapy, JNCI, 90, 12 Jun. 17, 1998.). The review discusses in detail the state of the prior art regarding major drugs, clinical applications and details regarding mechanism of action in the field of photodynamic therapy.

In a study on the mode of killing by photodynamic therapy, the present inventors discovered that agents localizing mainly in mitochondria catalyze damage to the mitochondrial membrane. This localization results in the release of cytochrome c into the cytosol. This phenomena has been shown to be a signal for cell death by a process known as apoptosis.

Apoptosis is a programmed cell death mechanism that is triggered either by an inherent genetic process or by cell damage. Release of cytochrome c after photodamage mimics the effect of natural or traumatic processes that elicit apoptotic cell death. The process involves activation of a series of enzymes that ultimately fragment cellular DNA in small vessicles that are subsequently engulfed by adjoining cells. Apoptotic death is therefore different from necrosis which involves loss of the outer membrane of the cell, resulting in the release of cellular DNA and lysosomal enzymes into the tissues, ultimately resulting in an inflammatory response.

With regard to the actual delivery of photodynamic therapy, the photosensitizing agent is injected into the blood stream and absorbed by cells all over the body. The agent remains in cancer cells for a longer time than it does normal cells. When the treated cancer cells are exposed to light, such as laser light, the photosensitizing agent absorbs the light and produces the active form of oxygen described above but destroys the treated cancer cells. Based on prior art methods of therapy, light exposure must be timed carefully so that it occurs when most of the photosensitizing agent has left healthy cells but is still present in the cancer cells.

The laser light used can be directed through a fiber optic (a very thin glass strand). The fiber optic is placed close to the cancer to deliver the proper amount of light. The fiber optic can be directed through a bronchoscope into the lung for treatment of lung cancer or through an endoscope into the esophagus for the treatment of esophageal cancer.

In principle and in practice, an advantage of photodynamic therapy is that it causes minimal damage to healthy tissue. However, because the laser light currently in use cannot pass through more than 3 cm. of tissue such therapy is mainly used to treat tumors on or just under the skin or on the lining of internal organs.

Photodynamic therapy makes the skin and eyes sensitive to light for about six weeks or more after treatment. Patients are advised to avoid direct sunlight and bright indoor light for at least six weeks. If patients must go outdoors, they are advised to wear protective clothing, including sunglasses. Even with these precautions, skin can become blistered, red or swollen. Other temporary side effects are related to the treatment of specific areas and can include coughing, trouble swallowing, abdominal pain, and painful breathing or shortness of breath.

In December, 1995, the U.S. Food & Drug Administration (FDA) approved a photosensitizing agent called porfimer sodium, sold under the trade name Photofrin®. Photofrin is used to relieve symptoms of esophageal cancer that is causing an obstruction and for esophageal cancer that cannot be satisfactorily treated with lasers alone. In January, 1998, the FDA approved porfimer sodium for the treatment of early nonsmall cell lung cancer in patients for whom the usual treatment for lung cancer are not appropriate.

Presently, the National Cancer Institute and other institutions are supporting clinical trials (research studies) to evaluate the use of photodynamic therapy for several other types of cancer, including cancers of the bladder, brain, larynx, an oral cavity.

It is desirable to develop photodynamic therapy which minimizes the aforementioned side effects, that is decreases persistent skin photosensitization. It would also be desirable to be able to achieve this goal utilizing means which are already well known in the art and represent a high degree of safety.

### SUMMARY OF THE INVENTION

In accordance with the present invention, there is provided a method of potentiating the phototoxicity of photodynamic therapy by co-administering a photosensitizing agent with a photodynamic potentiator which is a member of the group consisting of ursodeoxycholic acid (UDCA) and analogues and conjugates thereof having phototoxicity potentiating effect, allowing for retention of the co-administered agent and acid in a target tissue, and then irradiating the target tissue. The present invention further provides a tool for potentiating apoptosis consisting of the photosensitizing agent and a non-toxic photodynamic potentiator. The present invention further provides a method of potentiating drug induced apoptosis by administering the photosensitizing agent and decreasing the threshold of responsiveness of a target tissue to a photokilling effect of the photosensitizing agent.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a chart demonstrating UDCA enhancement of SnT2 PDT cell killing;

FIG. 2 is a bar graph showing UDCA enhancement of SnT2 PDT-mediated caspase-3 activation; and

FIG. 3 is a bar graph showing the influence of time of UDCA exposure on enhancement of SNT2 PDT-mediated caspase-3 activation.

### DETAILED DESCRIPTION OF THE INVENTION

Generally, the present invention provides a method of potentiating the phototoxicity of photodynamic therapy by co-administering a photosensitizing agent with a photodynamic potentiator.

More specifically, the term “phototoxicity” is used herein to mean the killing of cells during irradiation of the cells wherein the cells have retained therein a photosensitizing agent. Photodynamic therapy, as explained in the background art section, is the therapeutic use of phototoxicity involving photosensitization of target tissues by any of the series of photosensitizing agents.

The agents, referred to herein as “photosensitizing agents”, preferably have a porphyrin-like structure to localizing tumors. Utility of a porphyrin to help in the early diagnosis of previously undiscovered neoplasms in a patient was recognized. Policard, A. Etudes sur les aspects offerts par des tufmeurs experimentales examine' es a la lumie re de Wood. Compt. Rend. Sos. Biol. 91: 1423 (1924). To this day, the reasons for the localization of the porphyrin-like agents in the neoplastic tissues is not fully understood. It is also known that photosensitizing agents show an affinity for atherosclerotic plaque and a class of retinal blood vessels that lead to macular degeneration. Accordingly, the present invention finds utility in the treatment of these diseases.

Among the non-porphyrin structures are a series of Texaphyrins developed by Jonathan Sessler at the University of Texas. These are currently being marketed by Pharmacyclics (Sunyvale Calif.). The texaphyrin structure is somewhat similar to the porphyrins, but has an expanded ring system. Some phthalocyanines have also been suggested as sensitizers; these have a porphyrin-like ring system, but with N replacing C in the structures that join the pyrrole rings together. None of these has as yet made a clinical impact, but a silicon phthalocyanine is being tested by the NIH (developed at Case Western Reserve University). This agent is said to have a mitochondrial target. It is possible that structures not based on the porphyrin or porphyrin-like systems will eventually be developed.

The preferred photosensitizing agent for use in the course of the present invention is Photofrin® (product of Sanofi Pharmaceuticals, Inc., New York, N.Y.). Photofrin is porfimer-sodium and is administered by injection. The compound is a photosensitizing agent used in photodynamic therapy of tumors. Photofrin is not a single chemical entity, but rather is a mixture of oligomers formed by ether and ester linkages of up to eight porphyrin units. The cyotoxic and anti-tumor actions of Photofrin are light and oxygen dependent.

Photodynamic therapy utilizing photosensitizing agents such as Photofrin is a two stage process. The first stage is intravenous injection of the drug. Clearance from a variety of tissues occurs over 40 to 72 hours, but tumors, skin, and organs of the reticuloendothelial system (including liver and spleen) retain such drugs for longer period of time. Illumination with laser light, preferably at 630 nm constitutes the second stage of therapy.

Tumor selectivity and treatment occurs through a combination of selective retention of the drug and selective delivery of the light. Cellular damage caused by the drug is a consequence of the propagation of radical reactions. Radical initiation can occur after a drug absorbs light to form a porphyrin excited state. Spin transfer from the drug to molecular oxygen can then generate a singlet oxygen. Subsequent radical reactions form super oxide and hydroxyl radicals. Tumor death also occurs through ischemic necrosis secondary to vascular occlusion that appears to be partly mediated through thrombaxane A₂ release. The laser treatment induces a photo chemical but not a thermal effect. The necrotic reaction and associated inflammatory response can evolve over several days.

In accordance with the present invention, the photosensitizing agent is co-administered with a photodynamic potentiator. The term “photodynamic potentiator” is used herein to mean a non-toxic compound which increases the efficacy of a dose of photosensitizing agent. That is, the photodynamic potentiator decreases the threshold of responsiveness of the photosensitizing agent to irradiation. Accordingly, in a dose responsive manner, co-administration of the photodynamic sensitizer with the photosensitizing agent results in the need for less of the photosensitizing agent to achieve the target cell killing effect. Concomittant with the potentiation of phototoxicity is the need for less porphyrin to obtain a desired target cell kill. Accordingly, less of the photosensitizing agent accumulates in non-target tissue, such as normal skin, non-target vessels, and the like. The practical result is the reduction in the dose of the photosensitizing agent needed for a therapeutic effect which alleviates the persistent skin photosensitization and various other adverse effects requiring patients to avoid bright lights for extended periods of time. This decreased in persistent skin photosensitization may not be completed alleviated, but rather significantly alleviated.

The photodynamic potentiators of the present invention are preferably selected from the group, including ursodeoxycholic acid (UDCA) and analogs and conjugates thereof having the phototoxicity potentiating effect. The structure of UDCA and its close structural relative, deoxycholic acid are shown below.

UDCA, sold under the trade name URSO® (product of Axcan Pharma U.S. Inc., Minneapolis, Minn. and others) is a bile acid presently available as 250 mg film-coated tablets for oral administration. UDCA or URSO diol is a naturally occurring bile acid found in small quantities in normal human bile and in larger quantities in the biles of certain species of bears. It is a bitter tasting white powder consisting of crystaline particles freely soluble in ethanol and glacial acetic acid, slightly soluble in chloraform, sparingly soluble in ether, and practically insoluble in water. UDCA is normally present in a minor fraction of the total bile acids of humans (about 5%). Following oral administration, the majority of UDCA is absorbed by passive diffusion but its absorption is incomplete. Once absorbed, the compound undergoes hepatic extraction to the extent of about 50% in the absence of liver disease. In the liver, it is conjugated with glycine or taurine, then secreted into the bile. The conjugates are absorbed in the small intestine by passive and active mechanisms. The conjugates can also be deconjugated in the ileum by intestinal enzymes. UDCA tablets are presently indicated for the treatment of patients with primary billilar cirrhosis.

To date, there have been no reports concerning the use of UDCA to enhance the phototoxicity of photodynamic therapy and specifically regarding the co-administration of UDCA analogs or conjugates, with a photosensitizing agent. Rather, procedures that have been suggested for the use of UDCA involve hyperbaric oxygen or hyperthermia, both complex procedures with unknown adverse effects. UDCA has been in medical practice for a quite long period of time, mainly for the dissolving of gallstones and for the treatment of certain liver diseases.

Drugs other than the photodynamic potentiators of the present invention can promote the photokilling by photosensitizing agents such as Photofrin. These include the protein kinase C inhibitors staurosporin and chelerythrine. These are toxic agents and cannot be used clinically. Applicants have conducted studies on the mode of killing by Photofrin and have found that agents localizing mainly in mitochondria catalyze damage to the mitochondrial membrane. This results in the release of citochrome c into the cytosol. This has been shown to be a signal for cell by a process known as apoptosis.

Apoptosis is a programmed cell-death mechanism that is triggered either by an inherent genetic process or by cell damage. Release of citochrome c after photo damage mimics the effect of natural or traumatic processes that elicit apoptotic cell death. The process involves activation of a series of enzymes that ultimately fragment cellular DNA into small vessicles that are subsequently engulfed by adjoining cells. Apoptotic death is therefore different from necrosis which involves loss of the outer cell membrane of the cell, resulting in release of cellular DNA and lysosomal enzymes into the tissues and an inflammatory response.

During applicant's studies on agents that can affect the release of cytochrome c after photodamage to mitochondria, applicants discovered that the biosalt UDCA promoted cell killing by a given dose of Photofrin. This discovery has profound implications with regard to the clinical use of photosensitizing agents since 1) UDCA has been used for many years for dissolving gallstones and for the treatment of liver disease and is therefore known to be a safe drug, and 2) promotion of the Photofrin affect can result in a reduction of the dose of photosensitizing agent, or enhanced killing of cells during irradiation. These two factors are critical. The use of the photosensitizing agent, such as Photofrin which is approved by the FDA, is accompanied by persistent skin photosensitization, and thus has an adverse affect discussed above which requires patients to avoid bright lights for as long as four to six weeks after therapy. A reduction in the dose of the photosensitizing agent needed for a therapeutic effect, can alleviate this problem. Moreover, the photokilling affect of the photosensitizing agent derives from cells receiving a sufficient level of drug and of light for apoptosis to occur. Any adjuvant treatment that decreases the threshold of responsiveness, such as the photodynamic potentiators of the present invention, is expected to promote efficacy.

Administration of Photosensitizing Agent and Photodynamic Potentiator

The photosensitizing and photodynamic agents of the present invention are co-administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art.

In the method of the present invention, the drugs can be administered in various ways. It should be noted that the drugs can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic sold or liquid fillers, diluents or encapsulating material not reacting with the active ingredients of the invention.

When administering the drugs parenterally, the drugs will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such as cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the drugs can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the drugs utilized in the present invention can be administered orally to the patient. Conventional methods such as administering the compounds in tablets, suspensions, solutions, emulsions, capsules, powders, syrups and the like are usable. Known techniques which deliver the drugs orally or intravenously and retain the biological activity are preferred.

In one embodiment, the drugs can be administered initially by intravenous injection to bring blood levels of drugs to a suitable level. The patient's levels are then maintained by an oral dosage form, although other forms of administration, dependent upon the patient's condition and as indicate above, can be used.

The quantity of drugs to be administered will vary for the patient being treated and will vary from about 100 ng/kg of body weight to 100 mg/kg of body weight per day and preferably will be from 10 μg/kg to 10 mg/kg per day.

More specifically, and preferably, Photofrin is administered in the two stage process discussed above. The first stage of photodynamic therapy is intravenous injection of Phorofrin at 2 mg/kg. UDCA is presently administered as 250 mg film coated tablets, delivered orally. It is expected that the dosage of Photofrin will be decreased with the administration of increased amounts of UDCA and/or its conjugats and analog. Illumination with laser light 40-50 hours following injection with Photofrin constitutes the second stage of the therapy. The second laser light application can be given 96 to 120 hours after injection, proceeded by general debridement of residual tumor.

Patients may receive a second course of treatment a minimum of thirty days after the initial therapy and up to three courses of treatment can be given, each separated by a minimum of thirty days.

Photofrin should be administered as a single slow intravenous injection over three to five minutes at 2 mg. per body weight. The product must be protected from bright light and used immediately once reconstituted.

Photofrin has been found effective in at least the treatment of esophageal cancer and endobronchial cancer as well as the treatment of other cancers.

The present invention also provides a tool for potentiating apoptosis, consisting of the photosensitizing agent and the non-toxic photodynamic potentiator. As discussed above, apoptosis can be initiated and potentiated in various systems, outside of therapeutic uses, in accordance with the present invention. Apoptosis in cell lines as well as in situ environments can be potentiated and used as a tool in the laboratory, as well as the clinic. Thus, the present invention provides a method of potentiating drug induced apoptosis by the administration of the photosensitizing agent and then decreasing the threshold of responsiveness of a target tissue to the photokilling effect of the photosensitizing agent.

The following experimental data demonstrates the utility of the present invention. Specifically, the data shows that increasing the amount of the photodynamic potentiator administered decreases the time of irradiation needed for the target tissue to produce a similar effect. Further, increasing the amount of photodynamic potentiator will decrease the dose of photosensitizing agent required to maintain an effective photokilling effect of the photosensitizing agent. Accordingly, therapeutic co-administration of the drugs in accordance with the present invention can decrease skin photosensitization by allowing for a decreased amount of the photosensitizing agent to be required.

### EXAMPLES

General Methods:

Cell lines. Studies have been carried out with the murine leukemia L1210 and murine hepatoma hepa 1c1c7 cell lines along with primary rat hepatocyte cultures. Except for the latter, cells are grown in tissue culture. The L1210 cell line is grown in suspension culture in Fischer's medium containing 10% horse serum. The murine hepatoma 1c1c7 cell line was obtained from Dr. J. Whitlock, Jr. (Stanford University, CA). Cells were cultured on either commercially available plastic tissue cutureware, or poly-L-lysine coated glass coverslips or discs, and grown at 37° C. in a-minimal essential medium (aMEM) containing 5% fetal bovine serum and antibiotics. Cultures were passaged by incubation with a trypsin+EDTA solution (0.25% trypsin, 1 mM EDTA in Hank's Balanced Salts Solution).

Photosensitization protocols. A series of photosensitizing drugs, together with their sites of action, are shown in Table 1:

TABLE 1

| Sensitizer | Target | Sensitizer | Target |
|---|---|---|---|
| Photofrin† | mitochondrai* | Hexyl pheophor- | mitochondria |
| | | bide | |
| Tin etiopurpurin | mito/lysosomes | ALA† | mitochondria |
| mTHPC | mitochondrai | Si-phthalocyanine | mitochondria |
| Lu-texaphyrin | lysosomes | Npe6 | lysosomes |
| BPD†‡ | mitochondria | | |

*Mainly mitochondria, may be some outer membrane photodamage
†FDA approval for some indications
‡Benzoporphyrin derivative; approved by FDA for therapy of macular degeneration.

A group of five agents concentrates in the mitochondria: ALA (this pro-drug, aminolevulinic acid, is converted by cellular enzymes into protoporphyrin), HPPH (a hexyl pheophorbide ester), CPO (a porphycene), mTHPC (meta-hydroxyphenyl porphine) and Photofrin (a mixture of porphyrin-related structures). The tin purpurin SnET2 and the aluminum phthalocyanine AIPc localize in both mitochondria and lysosomes. Three agents localize in lysomes: LCP and NPe6 are both chlorins, LuTex is a lutetium texaphyrin. LCI is another chlorin that localizes in both the outer membrane and in lysosomes. MCP is a monocationic porphyrin that partitions only to the outer membrane. Of these agents, Photofrin and ALA currently have FDA approval for some indications, LuTex and SnET2 are in various stages of clinical trials.

Determination of PDT efficacy. Cell suspensions (7 mg/ml wet weight=2×10⁶ cells) or petri dishes containing 70%—confluent 1c1c7 cells or primary rat hepatocytes are incubated with photosensitizing agents, usually for 30 minutes at 37° C. Cells were washed free from extracellular drugs, and irradiated using 270 mJ/cm², a light dose that generally yields a 30% loss of cell viability. Cells were then incubated at 37° C. for an additional three to ten minutes to permit the released cytochrome c to begin activation of caspase-3, an enzymatic step involved in the apoptotic process. The cells were then lysed and levels of caspase-3 activity are assessed using a fluorescent plate reader. The substrate is DEVD-afc or DEVD-rhodamine 110. These substrates were cleaved by caspase-3 to form fluorescent products that were then detected. The plate readers take periodic readings of the emerging fluorescence, permitting a rate to be assessed. The general procedure involved quadruplicate samples, with the mean±standard deviation automatically plotted. Protein levels in the lysates were also determined, so that the net result was in terms of caspase-3 activity per mg protein per minute.

Photokilling was assessed by clonogenic assays involving the plating of known numbers of cells. After several days in cell culture, the number of resulting colonies reflected the number of viable cells that survive PDT. Freshly prepared suspensions of 1c1c7 cells were plated in 60 mm culture dishes at a density of 500 cells/dish. Approximately 18 hours later cultures were treated with PDT sensitizer and/or UDCA. After 30 minutes of incubation culture dishes were washed once with complete medium and then refed with complete medium. Refed cultures were subsequently irradiated and returned to incubators. Cultures were refed every third day, and stained with crystal violet once colonies were of sufficient size to score.

Treatment of cultures used for analyses of caspase activities. Two day old cultures of 1c1c7 cells (˜60-70% confluent) were treated with PDT sensitizer and/or UDCA for 30 minutes before being washed once and refed with complete medium. Cultures were subsequently irradiated and returned to the incubators for varying lengths of time before being harvested for caspase assays. In some studies, as noted in figure legends, UDCA was also added to culture media after irradiation. The procedures used for the harvesting of cultures and the assaying of caspases-3/7 have been described in detail (Reinders, J. J., Jr. and Clift, R. E. J. Biol. Chem. 274, 2502-2510, 1999). The assay measures the conversion of Ac-DEVD-AMC to the fluorescent product 7-amino-4-methyl coumarin. This same assay was used with L1210 cells, or a similar assay where the fluorophore was rhodamine-110.

To assess the effects of the different bile salts on PDT-induced caspase-3 activation and on photokilling, UDCA or any of its analogs were added during the initial incubation with the photosensitizing agent, after irradiation, or both. The concentration of the bile salts was varied from 10-100 μM. All bile salts were prepared as 100 mM solutions in 200 nM NaOH to minimize solvent effects. The single exception was lithocholic acid that must be dissolved in DMSO. When this agent was used, controls of DMSO alone were added to test for the effect of the solvent on PDT-induced cell killing.

### SUMMARY OF RESULTS

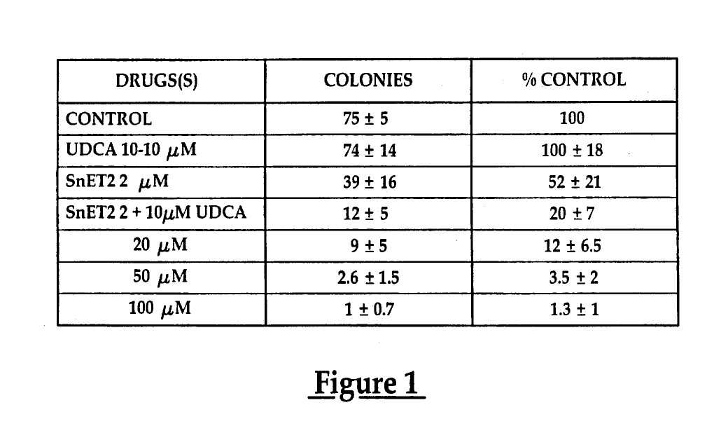

FIG. 1 shows 1c1c7 cultures plated 18 hours earlier that were exposed to varying concentrations of UDCA, and, as specified, the photosensitizing agent SnET2 (2-mM). After irradiation of 270 mJ/cm² at 630-700 nm, the plates were incubated and the colonies were counted four days later. The data represents the mean±a standard deviation of three to four plates.

FIG. 1 shows a clear dose dependent response resulting in a decrease of 12±5 colonies to 1±0.7 colonies (a decrease of 20% to 1.3%) with an increase in UDCA of 10 micromoles to 100 micromoles. No significant difference is seen between control and the addition of UDCA at the same varying concentrations.

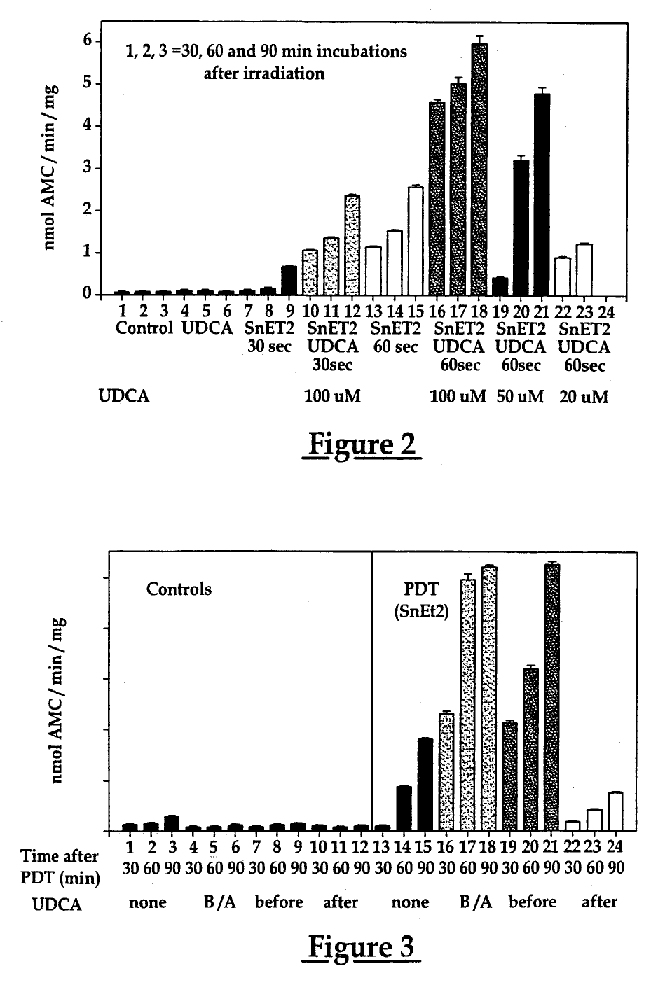

UDCA enhancement of SnT2 PDT-mediated caspase-3 activation is shown in FIG. 2. Two day old subconfluent 1c1c7 cultures were exposed to SnET2 (2 μM concentration and/or varying concentrations of UDCA as specified). After irradiation (270 mJ/Cm² at 630-700 nm), cells were harvested for preparation of extracts for caspase-3 (DEVDase) assays at varied times after irradiation. Data shown in FIG. 2 represent the mean±a standard deviation of four determinations. FIG. 2 shows a significant potentiation of caspase-3 activation after mitochondrial photo damage with increasing times of exposure to light. The data further shows a dose dependency, there being significant increase in potentiation with an increase in UDCA doses from 50 mM to 100 mM. Potentiation between photosensitizer alone and photosensitizer with photodynamic potentiator is significant, even at low light exposure and lower dose.

FIG. 3 demonstrates the influence of time of UDCA exposure on the enhancement of photosensitizing agent mediated caspase-3 activation. Two day old subconfluent 1c1c7 cultures were exposed to vehicle alone, SnET2 (2 mM), and/or 100 mM UDCA. Some cultures were irradiated (270 mJ/cm²). UDCA was present either before, after or both before and after irradiation. Cultures were harvested for preparation of extracts for caspase-3 (AEVDase DEVD) activation at varied times after irradiation. Data represent mean±standard deviation of four determinations. The chart shows no enhanced activation of the caspase-3 in controls. Data further shows that enhanced activation requires that UDCA present before irradiation. There is no significant difference between UDCA being applied after irradiation at any of the time intervals over none being administered. When UDCA is given either before and after or before, significant potentiation is obtained. Hence, it can be concluded that there is no enhanced activation of caspase-3 unless the UDCA is administered either with the photosensitizing agent or before the administration of the photosensitizing agent.

The above experiments demonstrate that the addition of as little as 10 mM UDCA to a cell culture before irradiation results in a marked decrease in the number of surviving cells after photodynamic therapy with SnET2. SnET2 is an agent that causes photodamage to mitochondria and lysosomes. This effect is accompanied by an enhanced activation of caspase-3 as assessed by the measurement of DEVDase activity.

In the hepatoma cell line, there is no evidence of promotion of photodynamic therapy efficacy when the lysosomal sensitizer Npe6 is used. Accordingly, the mechanism of action of the present invention appears to be related to mitochondrial photodamage. This is further supported by experiments in murine leukemia cells. In murine leukemia cells tested, the enhanced activation of caspase-3 by UDCA is observed only when mitochondrial or mitochondrial and lysosomal photodamage occurs. Agents whose mode of action is confined to lysosomes and/or the outer membrane appear to be minimally affected by the presence of the photodynamic potentiator. Applicants have observed some enhanced DEVDase activity by these agents and this result may be related to a minor photosensitization of mitochondria by the sensitizers that mainly target lysosomes.

An examination of a series of UDCA analogs indicates that the conjugates UDCA-taurine and UDCA-glycine are also effective in promoting photodynamic therapeutic photokilling. This is of potential importance since UDCA is known to conjugate with these amino acids in the liver of man, as discussed above. The very hydophobic UDCA analogs, the deoxycholic acid and lithcholic acid initiate apoptosis without a photodynamic effect. This likely results from a detergent-like effect on the mitachondrial membrane resulting in the loss of cytochrome c. Other less hydophobic analogs of UDCA also promote photodynamic induced killing by apoptosis. UDCA is a very likely agent for clinical trials since this drug is known to be safe for human administration.

Studies on primary hepatocytes show no enhanced photodynamic therapy induced apoptosis by UDCA. This is of importance since it suggests that the effect used by UDCA may be confined to neoplastic cell types.

In view of the above, any bioacid within an appropriate degree of hydrophobicity will potentiate the photodynamic effect.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

What is claimed is:

1. A method of potentiating the phototoxicity of photodynamic therapy by co-administering a photosensitizing agent with a photodynamic potentiator from the group consisting of ursodeoxycholic acid (UBCA) and analogs and conjugates thereof having the phototoxicity potentiating effect; allowing for retention of the co-administered agent and potentiator in a target tumors; and then irradiating the target tumors.

2. A method according to claim 1 wherein said co-administering step is further defined as administering a photosensitizing agent selected from the group consisting of photoactivated phorphyrins.

3. A method according to claim 1 further defined as increasing the amount of the photodynamic potentiator administered while decreasing time of irradiation of the target tumors.

4. A method according to claim 1 further defined as increasing the amount of photodynamic potentiator while decreasing the dose of photosensitizing agent while maintaining an effective photokilling effect of the potentiating agent.

5. A method according to claim 1 further defined as increasing the amount of photodynamic potentiator while decreasing skin photosensitization.

6. A method according to claim 1 wherein said co-admnistering step is further defined as co-administering a photosensitizing agent selected from the group which causes mitochondrial or mitochondrial lysosomal damage with the photodynamic potentiator.

7. A method according to claim 6 further including the step of affecting release of cytochrome c after photodamage to mitochondria resulting in apoptosis.

8. A tool for potentiating apoptosis consisting of a photosensitizing agent and a non-toxic photodynamic potentiator.

9. A method according to claim 8 wherein said photodynamic potentiator is selected from the group consisting of UDCA and analogs and conjugated thereof having the phototoxicity potentiating effect.

10. A method according to claim 8 wherein said photosensitizing agent is selected from the group consisting of photoactivated porphyrins.

11. A method of potentiating drug induced apoptosis by administering a photosensitizing agent and decreasing the threshold of responsiveness of a target tissue to a photokilling effect of the photosensitivity agent.
